(19) United States
(12) Patent Application Publication
(10) Pub. No.: US 2014/0271881 A1
(43) Pub. Date: Sep. 18, 2014

(54) ANTISEPTIC COMPOSITION

(71) Applicants: Jean-Claude Allart, Le Touquet (FR); Jean-Marie Lefevre, Amiens (FR); Jacques Peyrot, Clermont-Ferrand (FR)

(72) Inventors: Jean-Claude Allart, Le Touquet (FR); Jean-Marie Lefevre, Amiens (FR); Jacques Peyrot, Clermont-Ferrand (FR)

(21) Appl. No.: 14/234,193

(22) PCT Filed: Aug. 2, 2012
(86) PCT No.: PCT/FR2012/051838

§ 371 Date: May 2, 2014

(30) Foreign Application Priority Data
Aug. 4, 2011 (FR) 1157144

(51) Int. Cl. A61K 33/38; A61K 31/722; A61K 45/06; A61K 31/728

(52) U.S. Cl. CPC A61K 33/38; A61K 31/728; A61K 31/722; A61K 45/06
USPC 424/489; 424/618

(57) ABSTRACT

The invention relates to an antiseptic composition. The composition comprises an association of at least silver metal, preferably in a micronised form, and hyaluronic acid or one of the salts thereof having a molecular weight of between 50 kDa and 2000 kDa. The invention can be especially applied to the field of veterinary medicine, for the treatment of skin wounds and the healing thereof.

The present invention relates to a novel composition with antiseptic and healing properties, and more particularly to a novel dermatological composition based on silver and on hyaluronic acid, of use in particular in veterinary medicine, for the treatment of skin wounds and the healing thereof.

The skin constitutes both a living anatomical barrier and an area of exchange between the body and its environment, the effectiveness of which conditions the maintaining of a good homeostatic balance. The skin is an actual organ comprising several integrated layers, ranging from the superficial layer, the epidermis, to the deeper layers, the dermis and the hypodermis, wherein each of these layers performs functions which enable the whole assembly to react and adapt to its environment.

The epidermis, mainly composed of keratinocytes, melanocytes and Langerhans cells, has a variable thickness according to the various parts of the body, and constitutes the external layer of the skin for protecting the body against its external environment. The dermis is the thickest layer, through which nerve fibers and blood vessels pass, and is composed mainly of collagen, elastin, proteoglycans and glycosaminoglycans, mainly synthesized by the dermal fibroblasts. The collagen fibers provide the mechanical strength and the texture of the skin, the elastin is responsible for its elasticity, and the glycosaminoglycans and the proteoglycans play a major role in terms of structure and moisturization of the skin. The deepest layer of the skin forms the hypodermis, containing the adipocytes which produce lipids ensuring the formation of a fat layer protecting the muscles, bones and internal organs against impacts.

In animals such as horses, dogs and cats, the skin also comprises highly developed pilosebaceous follicles which play an important role in protection and homeostatic equilibrium. The presence of the coat, which must be lubricated and protected, explains the important function of the sebaceous gland in the animal's physiological equilibrium. Keratinization disorders (genetic or acquired associated with the animal's life conditions), which are common in pets, are accompanied by weakening of the coat and the development of bacterial and fungal overpopulation. Furthermore, primary infections, dermatosis and wound superinfections, and parasitosis, are common. Certain anatomical areas in cats and dogs, such as the ears, the eyes, the mucosa-skin junction areas and the pads, are exposed and more commonly suffer from an infection.

In animals as in humans, skin wounds induce inflammatory healing phenomena which can be disrupted by the acute hypoxia caused by trauma and by the microbial pullulation triggered by the breaking of the skin. The effect of a bacterial overpopulation is to modify the conditions of the healing by delaying it or by blocking it, and by promoting the spread of an infection from the initial wound.

Most bacterial colonies transform into biofilms, a microbial survival membrane structure, diverting growth and healing factors by creating an inflammation which prevents the antimicrobial defense process from acting effectively. The bacteria of biofilms withstand most antibiotics and antiseptics, and it is therefore necessary to develop compositions capable of managing the microbial flora and of facilitating wound healing.

Silver is known to be an antiseptic and has been used for years, in particular in creams for treating neonatal infections and more widely as an effective antiseptic against aerobic and anaerobic Gram-positive and Gram-negative bacteria. The active form is the silver ion Ag⁺ which can be provided by various silver salts and complexes, such as silver nitrate, silver chloride or silver thiosulfate, or as various carboxylates such as the acetate, or sulfamide-derived complexes, for instance silver sulfadiazine. Examples of silver salts with anti-microbial properties are described in patent application US 2006051385.

These silver salts have been used since they are relatively easy to formulate in pharmaceutical form and their cost price is generally not very high. However, they exhibit toxicity by systemic resorption which limits their use, unless it is at low concentration, thereby greatly limiting their efficacy. Silver can also be used in metal form, but the cost price of compositions comprising silver metal is then very high, and, furthermore, the solubilization of silver metal is very difficult. However, although the known compositions based on silver, such as silver sulfadiazine, often exhibit good skin tolerance, they do not have any bactericidal advantage compared with reference antiseptics such as chlorhexidine, in particular on Gram-negative bacteria. Patent EP 241175 describes the combination of silver sulfadiazine with miconazole or clotrimazole in a broader-spectrum antimicrobial composition. Chlorhexidine and silver sulfadiazine have also been proposed in combination, more particularly as an antiseptic in the hospital environment for the prevention of infections, without providing any significant advantage (Hockenhull J C et al., Health Technol. Assess. 2008:12).

Hyaluronic acid is a natural polymer based on a disaccharide comprising D-glucuronic acid and N-acetylglucosamine units linked via glucosidic linkage. Its properties are very variable depending on its molecular weight. Hyaluronic acids with a high molecular weight, greater than 1000 kDa, are essentially used for promoting skin moisturization through the hydrophilic carbohydrate network that they form. Hyaluronic acids with a low molecular weight, less than approximately 50 kDa, can cross the barrier of the stratum corneum and stimulate the CD44 receptors responsible for hyaluronic acid neosynthesis in the dermis.

Hyaluronic acid, like collagen, is one of the main constituents of the extracellular matrix of the dermis and it plays an important role in cell growth and the maintenance of moisturization. During skin aging, a decrease in its concentration in the dermis is observed. Hyaluronic acid is a glycosaminoglycan which is often used in cosmetic and dermatological compositions, generally in the form of sodium hyaluronate, in particular for promoting moisturization, and for stimulating healing and the natural defenses of the skin. It is also used in cosmetic surgery for filling wrinkles, in medical treatments against arthrosis, and in ophthalmology. Various crosslinked or noncrosslinked hyaluronic acid derivatives which are capable of exhibiting good resistance to enzymatic degradation, and which can be used in particular in cosmetic compositions, are described, for example, in application WO 2011/080450.

It has also been proposed to combine hyaluronic acid or derivatives with chondroitin sulfate, as in application WO 2009/073437, with a C-glycoside derivative, as in application WO 2009/024719, or with a retinoid and an oligosaccharide, as in patent FR 2 894 827. Heavy metal salts of hyaluronic acid have also been proposed in antimicrobial compositions, as described in application WO 87/05517.

Patents DE 102007044583 and DE 102007044202 describe compositions combining colloidal silver with urea, optionally with the addition of hyaluronic acid or sodium hyaluronate, for treating skin wounds. Patent DE 102005008299 relates to a composition comprising silver, as an antiseptic, in combination with a moisturizing agent such as panthenol and hyaluronic acid. However, none of these documents gives any information as to the characteristics of the hyaluronic acid, in particular its molecular weight.

The object of the present invention is to develop a novel topical composition which has antiseptic and healing properties making it possible to effectively treat skin wounds against microbial proliferation and to promote healing, more particularly in animals.

A subject of the present invention is therefore a dermatological composition comprising a combination of at least silver metal and hyaluronic acid or one of the salts thereof.

A subject of the present invention is also an antiseptic and healing topical dermatological composition comprising a combination of at least silver metal and hyaluronic acid, or one of the salts thereof, having a molecular weight of between 50 kDa and 2000 kDa.

Finally, a subject of the present invention is a composition comprising a combination of at least silver metal and hyaluronic acid or one of the salts thereof, for use in the treatment of skin wounds and the healing thereof, more particularly in veterinary medicine.

The silver metal is preferably in micronized form, facilitating dispersion in a composition which may be hydrophilic or lipophilic.

According to one advantageous embodiment, the composition of the invention also comprises an acetylglucosamine derivative chosen from chitin and chitosan.

Such a composition is particularly useful in veterinary dermatology for the treatment of skin wounds by virtue of its antiseptic and healing properties.

The silver metal in micronized form can be easily dispersed in the composition of the invention, which may be hydrophilic or lipophilic, at concentrations sufficient for providing good antiseptic efficacy. The tests carried out with the compositions of the invention have demonstrated a very broad spectrum of antimicrobial activity, with a specific action on the anaerobic bacteria of biofilms, that the known antimicrobial silver salts do not make it possible to achieve, even in combination with an antiseptic such as chlorhexidine. The silver concentration is generally between 0.001% and 1.0% by weight relative to the total weight of the composition.

The silver metal used in the invention is advantageously in micronized form, the particle size being from approximately 1 to 20 μm, with a purity greater than 99.5%, and preferably greater than 99.8%, exhibiting an effective antibacterial and antimicrobial activity. An appropriate silver metal form consists of a porous structure which gradually releases the silver ions, for instance the microsponge structure of the product commercially available under the brand name MicroSilver BG.

According to the present description, the term “hyaluronic acid” is intended to mean hyaluronic acid in free form or in the form of an alkali metal or alkaline-earth metal salt thereof, for example sodium hyaluronate, potassium hyaluronate, calcium hyaluronate or magnesium hyaluronate, having an average molecular weight of between 50 and 2000 kDa, preferably between 100 and 1000 kDa and more preferentially between 300 and 500 kDa.

The hyaluronic acid that can be used in the invention is commercially available in various suitable forms according to the uses envisioned. It can be produced industrially in large amounts by extraction from animal tissues, such as rooster combs, or by bacterial fermentation, or else via a biotechnological process from vegetable substances, for example wheat.

Hydrolyzed hyaluronic acid or hyaluronic acid sodium salt, having a molecular weight of between 100, preferably 300, and 500 kDa can, for example, be used. The comparative tests carried out have shown that the best results are obtained with such acids, for instance those commercially available under the brand name PrimalHyal 300 having a molecular weight equal to 300 kDa and PrimalHyal 450 having a molecular weight equal to 450 kDa.

The compositions according to the present invention can comprise an effective amount of each of the active agents above, and for example between 0.05% and 2% by weight of hyaluronic acid, between 0.001% and 1% by weight of silver metal and between 0.1% and 15% by weight of chitosan or of chitin, relative to the total weight of the composition.

Chitosan, and where appropriate chitin, when combining with hyaluronic acid, have a protective crosslinked network effect which promotes the endogenous production of keratinocyte growth factors inducing fibroblast activity, increasing collagen production and accelerating the differentiation of fibroblasts into myofibroblasts.

The compositions according to the invention can also contain one or more secondary active agents which advantageously reinforce or supplement the activity of the combination of hyaluronic acid and silver metal, and are compatible, i.e. not capable of reacting with one another or of masking or limiting their respective effects.

The secondary active agents can be advantageously chosen from, for example, a healing agent, an ant-Inflammatory, an anti-infective and a vitamin such as vitamin A or E. The healing agent can be, for example, a zinc salt, or an oligogalactomannan-rich carob seed extract. The anti-inflammatory can be a polysaccharide such as Rhamnosoft® (Solabia) which inhibits cell adhesion and limits inflammatory reactions, or a Boswellia serrata gum extract (Soothex®) which acts by enzymatic inhibition of leukotriene synthesis. The supplementary anti-infective can be chosen from a silanol and an antimicrobial peptide formation activator such as methyl caproyl tyrosinate (Defensamide®).

As shown by the results set out hereinafter, the composition of the invention has a direct antiseptic activity on biofilms and increases the activity of innate immunity via β-defensins.

Bacterial samples taken from a chronic wound in animals, before any intervention and after application of the composition of the invention, have been placed in culture in an aerobic medium and in an anaerobic medium, and have been studied by microbial PCR (polymerase chain reaction), a technique which consists in specifically amplifying a DNA fragment so as to make it detectable, demonstrating a significant decrease in the number of bacteria during the treatment.

DNA microchip studies of the genomic expression of β-defensins have proved that their secretion increases under the action of the composition of the invention.

The studies carried out have shown that the compositions according to the invention have the effect of reinforcing the skin's natural defenses via the release of β-defensins-2 and -3 by keratinocytes, which limit microbial proliferation, in particular owing to their action on aerobic and anaerobic Gram-negative bacteria. Furthermore, the hyaluronic acid used has an anti-inflammatory action which limits painful reactions by blocking TRPV 1 and 3 receptors.

Clinical studies on animals suffering from traumatic or chronic wounds have demonstrated a considerable reduction in wound healing time in comparison with a reference product based on a combination of Centella asiatica having a healing action and essential oils of rosemary, of thyme and of lavender having an antiseptic action. The healing time, generally between 7 and 15 days for acute wounds, is reduced by 3 to 4 days with the composition of the invention. In the case of chronic wounds, the beginnings of healing is observed from day 5 with the composition of the invention, contrary to the usual compositions, such as sulfadiazine, which prove to be not very effective. The effectiveness of the healing is confirmed by means of a growth factor labeling study showing accelerated secretion of these factors.

The compositions according to the present invention may be in any of the usual galenic forms for topical application, in particular for external topical, application. They may be, for example, in the form of aqueous or aqueous-alcoholic solutions, micellar lotions, spray solutions, shampoos, dispersions, sera, wipes, patches, controlled-release dressings or tulle dressings, gels (aqueous, anhydrous or lipophilic), oleogels (lipid gels), ointments, suspensions, ionic or nonionic vesicular dispersions, liquid or semi-liquid emulsions (for example a milk), or solid or semi-solid emulsions. The emulsions may be of the oil-in-water (O/W) or water-in-oil (W/O) type, for example gels or creams.

The excipients and supports that can be used for preparing the compositions in accordance with the present invention are those commonly used in preparations for dermatological or veterinary use, and chosen according to the form of administration retained. By way of example, mention may be made of emulsifiers, thickeners, gelling agents, emollients, preservatives, solubilizing agents, suspending agents and also washing bases and fragrances.

The emulsifying agent can be chosen, for example, from high-molecular-weight carboxyvinyl polymers such as Carbopol®, polysorbates such as Tween 20® or Tween 60®, an alkyl polyglucoside such as Montanov®, and sorbitan esters and more particularly a sorbitan stearate, a sorbitan palmitate or a sorbitan laurate such as Arlacel®. It is also possible to use, as emulsifying agent, a derivative of stearic or palmitic acid, for example polyethylene glycol stearate, glyceryl stearate, PEG 100® stearate (for example Arlacel 165®), a steareth or a ceteareth, a fatty alcohol such as a stearyl, capryl or cetearyl alcohol, or else an emulsifiable silicone.

The gelling agents and thickeners are incorporated into the composition in order to improve the fluidity thereof. They can be chosen, for example, from polyacrylamides of the Carbopol type, acrylate/acrylic acid copolymers such as Aculyn®, crosslinked acrylates such as Carbopol Ultrez®, cellulose derivatives such as hydroxypropylcellulose, or natural gums such as xanthan gum.

Various preservatives can be used in the composition of the invention, such as phenoxyethanol, methyl para-hydroxybenzoate (methylparaben), ethyl para-hydroxybenzoate (ethylparaben), and Seppicide HB® which combines phenoxyethanol and para-hydroxybenzoates and has a broad antimicrobial spectrum.

The moisturizing agents and emollients used in the composition can be, for example, chosen from propylene glycol, glycerol, butylene glycol and shea butter, and also fatty alcohols. A suitable suspending agent is, for example, a clay such as bentonite or smectite.

The compositions in accordance with the present invention are prepared by the usual techniques, according to the administration form chosen, the desired amounts of hyaluronic acid, or of salts thereof, being mixed with the silver, and optionally the chitin or the chitosan, and the supports and excipients, in a physiologically acceptable medium.

For the purpose of the present invention, the term “physiologically acceptable” is intended to mean supports and excipients of a type commonly used in dermatological compositions in human and veterinary medicine, which are neutral with respect to the active ingredients used, which do not have a toxic effect and which do not cause any harmful side effect on the skin.

For example, in the case of a cream, a fatty phase can be dispersed in an aqueous phase in order to obtain an oil-in-water emulsion, or vice versa in order to prepare a water-in-oil emulsion, the active ingredients being in either phase.

The composition of the invention, for example in the form of a cream, is preferably applied two to three times a day to the area of the skin requiring the treatment, for a period of time which can range from a few days to four weeks depending on the seriousness of the affection. It is quite particularly of use for the treatment of traumatic acute wounds or chronic wounds in animals such as horses, dogs, cats and rabbits.

The following examples illustrate the invention in greater detail without limiting the scope thereof. In all the examples of compositions which follow, the parts are expressed by weight, unless otherwise indicated.

### EXAMPLE 1

An aqueous solution is prepared by mixing, at ambient temperature, the constituents indicated hereinafter.

| | |
|---|---|
| Micronized silver metal (10 μm) | 0.05 |
| Hyaluronic acid MW 300 | 0.20 |
| Chitosan | 3.50 |
| PEG 80 sorbitan laurate | 5.00 |
| Smectite | 0.20 |
| Demineralized water | q.s. 100.00 |

The micronized silver metal is a product that is commercially available under the brand name MicroSilver BG® (Impag). The hyaluronic acid has a molecular weight of 300 kDa (PrimalHyal 300®, Soliance).

### EXAMPLE 2

A fluid emulsion (milk) having the composition hereinafter is prepared by means of a conventional, technique.

| | |
|---|---|
| Micronized silver metal (10 μm) | 0.02 |
| Hyaluronic acid MW 300 | 0.10 |
| Chitosan | 2.50 |
| Polyethylene glycol stearate | 2.00 |
| Caprylyl methicone | 5.50 |
| Demineralized water | q.s. 100.00 |

The micronized silver and the hyaluronic acid are identical to those of example 1.

### EXAMPLE 3

An antiseptic cream is prepared by means of the usual techniques by successively mixing the phases containing the constituents indicated hereinafter, phases A and B being mixed while hot (65-70° C.)

| Phase A | |
|---|---|
| Arlacel 165 ® | 5.00 |
| Lanette O ® | 2.00 |
| Shea butter | 2.00 |
| Isononyl isonanoate | 6.00 |
| Phase B | |
| Water | q.s. 100.00 |
| Carbopol Ultrez ® | 0.25 |
| Phase C | |
| Glycerol | 5.00 |
| Micronized silver metal (MicroSilver) | 0.10 |
| Phase D | |
| Hyaluronic acid (PrimalHyal 300) | 0.05 |
| Water | 10.00 |
| Phase E | |
| Preservative | 0.80 |
| Fragrance | 0.25 |

The pH of the composition is adjusted to 5.8 by adding 32% sodium hydroxide.

This cream can be used by application to the areas of the skin to be treated, once or twice a day for a period of time which is determined by the practitioner, and which can range from one to three weeks according to the affection treated.

### EXAMPLE 4

An antiseptic cream is prepared by means of the usual techniques by successively mixing the phases containing the constituents indicated hereinafter.

| Phase A | |
|---|---|
| Cetearyl Alcohol & Cetearyl Glucoside | 4.00 |
| (Montanov 68 ®) | |
| Cetearyl alcohol | 2.50 |
| Cetiol RLF ® | 3.00 |
| Glyceryl stearate | 3.00 |
| Oleic sunflower oil | 6.00 |
| Phase B | |
| Water | 70.56 |
| Carbopol Ultrez 21 ® | 0.10 |
| Xanthan gum | 0.10 |
| Propanediol | 2.00 |
| Preservative | 1.00 |
| Phase C | |
| Sodium hydroxide (aqueous solution at 32%) | 0.04 |
| Phase D | |
| Glycerol | 2.00 |
| Micronized silver metal (MicroSilver) | 0.10 |
| Phase E | |
| Hyaluronic acid (PrimalHyal 450) | 0.10 |
| Water | 5.00 |
| Phase F | |
| Chitosan succinamide | 0.50 |

This cream is prepared by mixing the successive phases, like that of the previous example.

### EXAMPLE 5

A clinical study of the effect of the combination used in the present invention was carried out on 6 dogs carrying inflammatory pathological conditions having led to acute traumatic or chronic wounds caused by lesion due to scratching with delayed healing.

The treatments carried out previously, in particular with sulfadiazine, had proved to be ineffective.

The composition of the invention was applied in spray lotion form, twice a day, directly onto the wound to be treated. The results were observed 1 (D1), 2(D2), 3(D3), 5(D5) and 7(D7) days after the 1^{st} application.

These results are given in the table hereinafter.

| Case | Pathological condition | Clinical observations |
|---|---|---|
| 1 | Deep pyodermatitis | Effective healing at |
| | | D5 |
| 2 | Delayed healing following an | Resumption of healing |
| | exeresis | at D3 |
| 3 | Wound from licking following | Improvement at D2 |
| | injury caused by a spikelet | Healing at D7 |
| 4 | Recurrent interdigital | Improvement at D2 |
| | pyodermatitis | Healing at D7 |
| 5 | Surgical wound superinfected by | Improvement at D2 |
| | licking after amputation for | 80% healing at D5 |
| | tumor | |
| 6 | Multiple excoriations associated | Improvement from the |
| | with atopic chronic pruritus | first application |

A study of the degrees of re-epithelialization and of contraction of traumatic wounds was carried out on the limbs of six horses.

On each horse, one wound was treated and another was untreated, and the change was measured each day for 14 days, in the morning and in the evening for 36 hours and then once a day. At the end of the study, a bacterial sample was taken.

The treatment consisted of the application of the cream) of example 4, in the morning and in the evening for 36 hours, and then once a day.

Histopathological analysis on Hemalun-Eosin-Safran staining: the beginnings of re-epithelialization are observed at day 3 on the treated wounds, but not on the control wounds. At day 7, the wounds are still open but are retracted, and epithelial proliferation is visible, with the beginnings of keratinization. At day 14, the wound is retracted and the surface epidermis is made completely or almost completely continuous by the newly formed epithelium.

Histomorphometric Study:

A microscope-associated software for shape recognition and measurement of distances and surface areas (Axiovision 4.6.1) was used. Measurements of wound width (MES1 interepithelial distance and MES2 intraepithelial distance), of newly formed epithelium length (MES3) and of re-epithelialization surface area (SUR) were carried out.

| | | | Untreated |
|---|---|---|---|
| | Measurement | Treated wounds | wounds |
| D2 | MES1 (μm) | 6326 | 6641 |
| | MES2 (μm) | 6326 | 6641 |
| | MES3 (μm) | 0 | 0 |
| | SUR (μm²) | 0 | 0 |
| D3 | MES1 (μm) | 5635 | 5934 |
| | MES2 (μm) | 5386 | 3358 |
| | MES3 (μm) | 343 | 500 |
| | SUR (μm²) | 8894 | 11884 |
| D7 | MES1 (μm) | 6398 | 7325 |
| | MES2 (μm) | 4817 | 6039 |
| | MES3 (μm) | 4234 | 3346 |
| | SUR (μm²) | 879989 | 651390 |
| D14 | MES1 (μm) | 3528 | 4550 |
| | MES2 (μm) | 71 | 87 |
| | MES3 (μm) | 3635 | 4905 |
| | SUR (μm²) | 922116 | 861126 |

The table below indicates the MES2/MES1 (2/1) and MES3/MES1 (3/1) ratios which are an indication of the change in the treated or untreated wound.

| | Ratio | Treated wounds | Untreated wounds |
|---|---|---|---|
| D2 | 2/1 | 1 | 1 |
| | 3/1 | 0 | 0 |
| D3 | 2/1 | 0.95 | 0.56 |
| | 3/1 | 0.06 | 0.08 |
| D7 | 2/1 | 0.75 | 0.83 |
| | 3/1 | 0.66 | 0.46 |
| D14 | 2/1 | 0.02 | 0.02 |
| | 3/1 | 1.03 | 1.08 |

It is observed that the wound is more contracted and the epithelialization more advanced at day 7 in the presence of treatment, which makes it possible to obtain, in 7 days, a re-epithelialization equivalent to that which requires 14 days without the treatment.

These results show that the combination of the invention is effective in the treatment of acute or chronic wounds which are traumatic post-burn wounds, infectious wounds, or wounds that are complications of certain dermatoses (atopy, parasitosis), or post-interventional wounds, and that it provides a solution in cases where other topical agents have proven to be ineffective, in particular sulfadiazine. The rapidity of response (observed most commonly in the first 48 hours) to the applications of the composition, which distinguishes it from the reference products such as sulfadiazine, where such a rapid response is never observed, should be emphasized.

1. A dermatological composition for use in veterinary medicine, said composition comprising:
a combination of at least silver metal and hyaluronic acid, or one of the salts thereof, having a molecular weight of between 50 kDa and 2000 kDa.

2. The dermatological composition as claimed in claim 1, wherein the hyaluronic acid has a molecular weight of between 100 kDa and 1000 kDa.

3. The dermatological composition as claimed in claim 2, wherein the hyaluronic acid has a molecular weight of between 300 kDa and 500 kDa.

4. The composition as claimed in claim 1, wherein the hyaluronic acid salt is selected from the groups consisting of alkali metal and alkaline-earth metal salts.

5. The composition as claimed in claim 4, wherein the hyaluronic acid salt is any one of sodium hyaluronate, potassium hyaluronate, calcium hyaluronate or magnesium hyaluronate.

6. The composition as claimed in claim 1, wherein the silver is in the form of metal particles of between 1 and 20 μm in size.

7. The composition as claimed in claim 1, wherein said composition also comprises either one of chitin or chitosan.

8. The composition as claimed in claim 7, wherein said composition comprises between 0.1% and 10% by weight of hyaluronic acid, between 0.001% and 1% by weight of silver metal and between 0.1% and 15% by weight of either one of chitosan or of chitin, relative to the total weight of the composition.

9. The composition as claimed in claim 1, wherein said composition is in a form for external topical application.

10. The composition as claimed in claim 1, wherein said composition also comprises one or more secondary active agents selected from the group consisting of a healing agent, an anti-inflammatory, an anti-infective and a vitamin.

11. A composition comprising:
a combination of at least silver metal and hyaluronic acid, or one of the salts thereof, having a molecular weight of between 50 kDa and 2000 kDa, said composition configured to application to skin wounds and healing thereof in veterinary medicine in.
